(19) United States
(12) Patent Application Publication
(10) Pub. No.: US 2026/0355105 A1
(43) Pub. Date: Nov. 12, 2026

(54) DUAL-RESPONSE DENTAL PLAQUE INDICATOR TABLET COMPRISING ANTHOCYANIN AND ENZYME FOR CATALYTIC DECOMPOSITION

(71) Applicant: Jiangxi Dentalbright Technology Co., Ltd, Jiujiang (CN)

(72) Inventors: Junyuan Qin, Nanchang (CN); Wei Min, Nanchang (CN); Huanjin Wu, Xiangxi Tujia (CN)

(21) Appl. No.: 19/708,012

(22) Filed: Jun. 15, 2026

(30) Foreign Application Priority Data
May 21, 2026 (CN) 202610705125.7

(51) Int. Cl. A61K 8/9789; A61K 8/37; A61K 8/44; A61K 8/49; A61K 8/66; A61K 8/67; A61K 8/73; A61Q 11/00

(52) U.S. Cl. CPC A61K 8/9789; A61K 8/375; A61K 8/44; A61K 8/498; A61K 8/66; A61K 8/678; A61K 8/738; A61Q 11/00; A61K 2800/45; A61K 2800/522

(57) ABSTRACT

A dual-response dental plaque indicator tablet comprising anthocyanin and enzyme for catalytic decomposition includes natural anthocyanin, color auxiliary agent, antioxidant, metal chelating agent, coating matrix, coating solution, enzyme for catalytic decomposition, disintegrant, forming agent, sweetener, flavoring agent and lubricant. According to the disclosure, the pH-dependent color change characteristics of anthocyanins are utilized, which appear red in acid-producing plaque areas (pH 4.5-5.5) and blue-violet in healthy regions. Meanwhile, the extracellular polysaccharide of plaque is hydrolyzed by the enzyme for catalytic decomposition, which indicates the disintegration process of plaque cooperatively, and the color returns to blue-violet after cleaning, indicating that cleaning is completed.

### CROSS-REFERENCE TO RELATED APPLICATIONS

This application claims priority to Chinese Patent Application No. 202610705125.7, filed on May 21, 2026, which is hereby incorporated by reference in its entirety.

### TECHNICAL FIELD

The present disclosure relates to the technical field of oral care, and in particular to a dual-response indicator tablet for dental plaque detection and degradation and a preparation method thereof.

### BACKGROUND

Dental plaque is a bacterial biofilm attached to tooth surfaces, composed of bacteria, intercellular substances, desquamated epithelial cells and food debris. Dental plaque is a core factor inducing oral diseases such as dental caries, gingivitis and periodontitis. Therefore, effective identification and removal of dental plaque are of great significance for maintaining oral health.

Dental plaque is colorless and invisible to the naked eye, and plaque disclosing agents produce color development on dental plaque. Currently, commonly used plaque disclosing agents mainly include basic fuchsin solution, erythrosine solution and anthocyanin solution. Among them, synthetic colorants such as erythrosine and brilliant blue are widely applied, featuring low price, good stability and favorable staining performance. However, impurities including by-products generated during synthesis may pose potential safety risks. Chinese patent document CN103908680A discloses a dental plaque detection agent using anthocyanins as colorants, with the anthocyanin content of 4%-6%. During use, a cotton swab needs to be dipped into the detection agent and applied to teeth. Nevertheless, this detection agent is in liquid form and has a complicated application process. In addition, the document also indicates that anthocyanins, as natural colorants, have technical limitations such as high cost, instability, easy precipitation, poor staining power and peculiar odor.

In the field of plaque indicator preparations, Chinese patent CN105288661A discloses a dental plaque staining tablet adopting edible phycoerythrin as a colorant, which is prepared by tableting auxiliary materials such as microcrystalline cellulose, dextrin and magnesium stearate. This product is used in the form of a solid tablet and easy to carry, but the synthetic colorant phycoerythrin adopted has no pH-responsive color changing function.

In terms of enzymatic degradation of dental plaque, Chinese patent CN113749969A discloses an oral care composition containing various enzymes including 0.5%-3% lysozyme and 0.1%-2% dextranase, which effectively reduces dental plaque. In addition, patent application CN 118873456A discloses an essence for disclosing and degrading dental plaque, which includes sodium bicarbonate, enzyme composition (lysozyme, alkaline protease, amylase, alkaline lipoxidase), antibacterial composition and the like, and has dual functions of disclosing and degrading dental plaque. However, the disclosing effect of the product mainly relies on bubbles generated by foaming of sodium bicarbonate to indicate plaque areas, without color indication. Patent application CN118384057A discloses an oral care gel for color development and degrading dental plaque, which combines plaque degrading enzymes with plant bacteriostatic systems, yet it also achieves color development by virtue of colorants, and no pH-responsive mechanism is mentioned.

In terms of the stability of anthocyanins, academic studies have clearly pointed out that anthocyanins are susceptible to factors such as pH value and temperature, resulting in poor stability. This is a major technical obstacle to the application in dental plaque indicators. Relevant studies show that anthocyanins degrade and fade significantly at a pH range of 4 to 5, and are also prone to degradation at a temperature range of 50-60 °C. Other review reports indicate that methods including acylation, copigmentation and encapsulation may effectively improve the stability and biological activity of anthocyanins. However, in the field of dental plaque indicator tablets, no publicly reported technical solution has systematically applied the above stabilization strategies to anthocyanins to enhance the storage stability of products.

In conclusion, the prior art has the following deficiencies. First, although the color reaction of anthocyanins as pH-responsive colorants to the acidity of plaque microenvironment is well known, it remains a technical difficulty to effectively improve the stability of anthocyanins in indicator tablets and overcome the problem of discoloration during storage. Second, the plaque indicating function and degradation function of existing products are mostly separated into different products or dosage forms, and integrated solutions are lacking. Third, the existing dual-function products fail to form a complete closed-loop interaction of “color indication—enzymatic degradation—color feedback after cleaning”, and cannot dynamically reflect the elimination of dental plaque. Targeting the above problems, the present disclosure provides a dual-response dental plaque indicator tablet with both plaque indicating and degradation functions and stable color development.

### SUMMARY

Aiming at the shortcomings of the prior art, the disclosure provides a dual-response dental plaque indicator tablet with the functions of plaque indication and decomposition, stable color development and safe use.

The technical scheme of the disclosure is as follows:
a dual-response dental plaque indicator tablet comprising anthocyanin and enzyme for catalytic decomposition includes the following components in percentage by mass: 0.5%-1.5% of natural anthocyanin, 0.2%-0.8% of color auxiliary agent, 0.05%-0.2% of antioxidant, 0.05%-0.2% of metal chelating agent, 20%-25% of coating matrix, 4%-10% of coating solution, 0.5%-5% of enzyme for catalytic decomposition, 10%-15% of disintegrant, 40%-60% of forming agent, 0.5%-5% of sweetener, 0.3%-1% of flavoring agent and 0.1%-1% of lubricant.

Further, the natural anthocyanin is selected from one or more of purple sweet potato anthocyanin, purple carrot anthocyanin, blueberry anthocyanin, elderberry anthocyanin, black wolfberry anthocyanin or purple cabbage extract.

Further, the color auxiliary agent is selected from one or more of rosmarinic acid, chlorogenic acid, tannic acid, quercetin or catechin.

Further, the antioxidant is selected from one or more of vitamin E, rosemary extract or ascorbyl palmitate.

Further, the metal chelating agent is selected from one or more of ethylenediaminetetraacetic acid, ethylenediaminetetraacetic acid disodium salt or ethylenediaminetetraacetic acid tetrasodium salt.

Further, the coating matrix is selected from one or more of cyclodextrin, pullulan, trehalose or polyvinyl alcohol.

Further, the coating solution is selected from one or more of glycerol, polyethylene glycol, propylene glycol or sorbitol solution.

Further, the enzyme for catalytic decomposition is selected from a composition of dextranase, allosteric enzyme and lysozyme, and a mass ratio of the dextranase to allosteric enzyme to lysozyme is 1.0-3.0:0.5-2.0:0.5-1.5.

Further, the disintegrant is selected from one or more of crospovidone, microcrystalline cellulose or sodium carboxymethyl starch.

Further, the forming agent is selected from one or more of glucose, polyethylene glycol, lactose, maltodextrin or sorbitol.

Further, the sweetener is selected from one or more of sucralose, aspartame, ammonium glycyrrhizinate or xylitol; the flavoring agent is selected from peppermint flavor or fruit flavor; and the lubricant is magnesium stearate.

The disclosure also provides a method for preparing the dental plaque indicator tablet, which includes the following steps:
step 1, mixing natural anthocyanin, color auxiliary agent, antioxidant, metal chelating agent, coating matrix and coating solution uniformly, performing vacuum drying, crushing and sieving with an 80-mesh sieve to obtain a powder of natural anthocyanin stabilized color developing system;
step 2, uniformly mixing the powder obtained in step 1 with enzyme for catalytic decomposition, disintegrant, forming agent, sweetener and flavoring agent, and then adding lubricant to continue mixing to obtain total mixed powder; and
step 3, compressing the total mixed powder into tablets to obtain the product.

Further, the temperature of vacuum drying in step 1 does not exceed 40 °C, and the water content after drying is less than 5%.

Further, the tableting parameters in step 3 are as follows: tablet weight is 0.45 g-0.50 g, diameter is 10 mm-11 mm, thickness is 5.5 mm-6.0 mm, tablet shape is round, and hardness is 60 N-90 N; the compressing environment temperature is 15 °C-25 °C, and the relative humidity is below 45% RH.

The action mechanism of the disclosure is that anthocyanins have pH-dependent color change characteristics, which are red in acidic environment (pH 4.5-5.5) and blue-violet in neutral environments (pH 6.5-7.2). The local dental plaque is acidic due to acid production by bacteria metabolism, and the anthocyanin changes from initial blue-violet to red after the indicator contact, which directly shows the position of highly active dental plaque. Meanwhile, the enzyme for catalytic decomposition hydrolyzes the extracellular polysaccharides of plaque, disintegrates the plaque structure, and restores the local pH to normal. The anthocyanins gradually return to blue-violet, indicating completion of cleaning.

The disclosure has the following beneficial effects.

First, double response and accurate indication: the disclosure integrates the functions of plaque detection and decomposition, realizes the accurate positioning of high-activity plaque through pH-responsive color change, and indicates the cleaning end point through color recovery, thus improving the oral cleaning efficiency.

Second, natural safety: natural anthocyanins and edible auxiliary materials are adopted, which has good biocompatibility and no safety concerns of synthetic colorants.

Third, stability improvement: the light resistance and oxidation resistance of anthocyanins are significantly improved by stabilizing the color developing system (color auxiliary agent, antioxidant, metal chelating agent and coating material), and the shelf life of products is prolonged.

Fourth, synergistic effect: enzymatic hydrolysis exposes more bacteria and acid-producing sites, which enhances the color response of anthocyanin, and anthocyanin itself has additional health benefits of anti-inflammation and inhibition of oral pathogenic bacteria adhesion.

### DESCRIPTION OF EMBODIMENTS

The present disclosure is further illustrated by the following embodiments.

### Embodiment 1

### Components and Preparation

(1) 200 g of purple sweet potato anthocyanins, 150 g of purple carrot anthocyanins, 62.5 g of rosmarinic acid, 87.5 g of quercetin, 20 g of vitamin E, 7.5 g of rosemary extract, 20 g of ethylenediaminetetraacetic acid disodium salt, 4500 g of cyclodextrin, 500 g of pullulan, 750 g of glycerol and 500 g of polyethylene glycol were mixed thoroughly. The resulting mixture was vacuum-dried at 40 °C or below until the water content was less than 5%, and then sieved through an 80-mesh sieve to obtain stabilized color-developing powder.

(2) The above powder was blended with 250 g of dextranase, 125 g of allosteric enzyme, 200 g of lysozyme, 2000 g of crospovidone, 1250 g of microcrystalline cellulose, 9727.5 g of lactose, 2000 g of maltodextrin, 1250 g of polyethylene glycol, 50 g of sucralose, 1000 g of xylitol and 150 g of fruit flavor to form a uniform mixture, and 200 g of magnesium stearate was subsequently added and mixed evenly.

(3) Tableting: the tablet weight was 0.45 g-0.50 g, diameter was 10 mm-11 mm, thickness was 5.5 mm-6.0 mm, tablet shape was round, and hardness was 60 N-90 N; the compressing environment temperature was 15 °C-25 °C, and the relative humidity was below 45% RH.

### Embodiment 2

(1) 175 g of purple carrot anthocyanins, 175 g of blueberry anthocyanins, 75 g of rosmarinic acid, 50 g of quercetin, 17.5 g of vitamin E, 10 g of ascorbyl palmitate, 30 g of ethylenediaminetetraacetic acid disodium salt, 4250 g of cyclodextrin, 750 g of trehalose, 750 g of glycerol and 250 g of polyethylene glycol were mixed thoroughly. The mixture was vacuum-dried at 40 °C or below until the water content was less than 5%, and then sieved through an 80-mesh sieve to obtain stabilized color-developing powder.

(2) The above powder was blended with 300 g of dextranase, 225 g of allosteric enzyme, 125 g of lysozyme, 2000 g of microcrystalline cellulose, 1000 g of sodium carboxymethyl starch, 1500 g of maltodextrin, 9942.5 g of sorbitol, 2000 g of polyethylene glycol, 25 g of sucralose, 1000 g of xylitol and 125 g of fruit flavor to form a uniform mixture, and 225 g of magnesium stearate was subsequently added and mixed evenly.

(3) Tableting was performed with the same process parameters as those in Embodiment 1.

### Embodiment 3

(1) 150 g of blueberry anthocyanins, 225 g of purple cabbage extracts, 87.5 g of rosmarinic acid, 37.5 g of quercetin, 25 g of vitamin E, 12.5 g of ascorbyl palmitate, 40 g of ethylenediaminetetraacetic acid disodium salt, 4750 g of cyclodextrin, 250 g of pullulan, 250 g of trehalose and 1250 g of glycerol were mixed thoroughly. The mixture was vacuum-dried at 40 °C or below until the water content was less than 5%, and then sieved through an 80-mesh sieve to obtain stabilized color-developing powder.

(2) The above powder was blended with 275 g of dextranase, 200 g of allosteric enzyme, 200 g of lysozyme, 2250 g of microcrystalline cellulose, 750 g of sodium carboxymethyl starch, 2000 g of maltodextrin, 9542.5 g of sorbitol, 1500 g of polyethylene glycol, 55 g of sucralose, 750 g of xylitol and 200 g of fruit flavor to form a uniform mixture, and 200 g of magnesium stearate was subsequently added and mixed evenly.

(3) Tableting was performed with the same process parameters as those in Embodiment 1.

### Comparative Example 1

(1) 175 g of purple sweet potato anthocyanins, 200 g of purple cabbage extracts, 5000 g of cyclodextrin, 500 g of pullulan, 500 g of glycerol and 750 g of polyethylene glycol were mixed thoroughly. The mixture was vacuum-dried at 40 °C or below until the water content was less than 5%, and then sieved through an 80-mesh sieve to obtain color-developing powder (without color auxiliary agent, antioxidant or metal chelating agent).

(2) The above powder was blended with 250 g of dextranase, 300 g of allosteric enzyme, 150 g of lysozyme, 2500 g of crospovidone, 500 g of sodium carboxymethyl starch, 9950 g of lactose, 1750 g of maltodextrin, 1250 g of polyethylene glycol, 75 g of sucralose, 750 g of xylitol and 150 g of fruit flavor to form a uniform mixture, and 250 g of magnesium stearate was subsequently added and mixed evenly.

(3) Tableting was performed with the same process parameters as those in Embodiment 1.

### Comparative Example 2

(1) 162.5 g of blueberry anthocyanins, 212.5 g of purple cabbage extracts, 75 g of rosmarinic acid, 100 g of quercetin, 35 g of vitamin E, 5 g of ascorbyl palmitate, 37.5 g of ethylenediaminetetraacetic acid disodium salt, 5500 g of cyclodextrin, 750 g of glycerol and 750 g of polyethylene glycol were mixed thoroughly. The mixture was vacuum-dried at 40 °C or below until the water content was less than 5%, and then sieved through an 80-mesh sieve to obtain stabilized color-developing powder.

(2) The above powder was blended with 2000 g of crospovidone, 1500 g of microcrystalline cellulose, 8772.5 g of lactose, 1500 g of maltodextrin, 2500 g of polyethylene glycol, 50 g of sucralose, 750 g of xylitol and 175 g of fruit flavor to form a uniform mixture, and 125 g of magnesium stearate was subsequently added and mixed evenly. No enzyme for catalytic decomposition is added in this step.

(3) Tableting was performed with the same process parameters as those in Embodiment 1.

### Comparative Example 3

(1) 200 g of erythrosine, 50 g of rosmarinic acid, 125 g of quercetin, 37.5 g of vitamin E, 7.5 g of ascorbyl palmitate, 32.5 g of ethylenediaminetetraacetic acid disodium salt, 5750 g of cyclodextrin, 500 g of pullulan, 250 g of glycerol and 1500 g of polyethylene glycol were mixed thoroughly. The mixture was vacuum-dried at 40 °C or below until the water content was less than 5%, and then sieved through an 80-mesh sieve to obtain color-developing powder (synthetic colorant adopted).

(2) The above powder was blended with 325 g of dextranase, 150 g of allosteric enzyme, 150 g of lysozyme, 2000 g of microcrystalline cellulose, 1250 g of sodium carboxymethyl starch, 1500 g of maltodextrin, 7822.5 g of sorbitol, 2000 g of polyethylene glycol, 75 g of sucralose, 1000 g of xylitol and 125 g of fruit flavor to form a uniform mixture, and 150 g of magnesium stearate was subsequently added and mixed evenly.

(3) Tableting was performed with the same process parameters as those in Embodiment 1.

### Performance Evaluation

### 1. Anthocyanin Stability Test

The indicator tablets prepared in Embodiments 1-3 and Comparative example 1 were placed in a non-shaded indoor environment at 15-30 °C. Samples were taken after 1 month, 2 months and 3 months respectively, and prepared into 1% aqueous solutions for color change observation. The evaluation grades were defined as follows: Grade A (basically no change), Grade B (slight change), Grade C (obvious change), Grade D (severe change). The test results are shown in Table 1.

TABLE 1
Anthocyanin stability test results

| Samples | 1 month | 2 months | 3 months |
|---|---|---|---|
| Embodiment 1 | A | A | B |
| Embodiment 2 | A | B | B |
| Embodiment 3 | A | A | B |
| Comparative | B | C | D |
| example 1 | | | |

The results showed that the storage stability of the stabilized indicator tablets of the present disclosure (Embodiments 1-3) was significantly better than that of the untreated sample in Comparative example 1. After three months, the samples in the embodiments still maintained good color developing performance, while severe color fading occurred in Comparative example 1. The results indicate that the synergistic stabilization system including color auxiliary agent, antioxidant, metal chelating agent and coating matrix adopted in the present disclosure effectively improves the light resistance and oxidation resistance of anthocyanins.

### 2. Dental Plaque Degradation Test

An in vitro dental plaque biofilm model was established using Streptococcus mutans (S. mutans ATCC 25175). Solutions of indicator tablets from Embodiments 1-3 and Comparative example 2 are added separately. After a 5-minute reaction, the residual amount of dental plaque was determined via crystal violet staining method (OD₅₇₀), and the plaque removal rate was calculated. The test results are shown in Table 2.

TABLE 2
Test results of dental plaque degradation performance

| | Residual plaque | Plaque removal |
|---|---|---|
| Samples | amount (OD₅₇₀) | rate (%) |
| Embodiment 1 | 0.18 ± 0.03 | 72.3 |
| Embodiment 2 | 0.15 ± 0.02 | 76.8 |
| Embodiment 3 | 0.16 ± 0.03 | 74.5 |
| Comparative | 0.52 ± 0.05 | 13.1 |
| example 2 | | |
| Blank control | 0.65 ± 0.06 | — |

The results showed that the plaque removal rate of the indicator tablets of the present disclosure exceeded 70% within 5 minutes, which was remarkably higher than that of Comparative example 2 without enzymes (13.1%). The composite enzyme for catalytic decomposition including dextranase, allosteric enzyme and lysozyme exhibits excellent degradation capacity against dental plaque biofilms.

3. pH-Responsive Color Change Test

Forty healthy volunteers aged 22 to 55 years were equally divided into four groups. Each group used the indicator tablets of Embodiments 1-3 and Comparative example 3 respectively for color change observation.

The application results were as follows: the tooth surfaces in the groups of Embodiments 1-3 initially appeared blue-violet, turned pink after about 30 seconds, and reverted to blue-violet after lasting for 2-7 minutes. By contrast, the group of Comparative example 3 containing synthetic colorant erythrosine remained red with no color variation. It is demonstrated that the present disclosure realizes the dual indicating functions of pH-responsive color development on dental plaque and color recovery after enzymatic degradation.

What is claimed is:

1. A dual-response dental plaque indicator tablet comprising anthocyanin and enzyme for catalytic decomposition, comprising following components in percentage by mass: 0.5%-1.5% of natural anthocyanin, 0.2%-0.8% of color auxiliary agent, 0.05%-0.2% of antioxidant, 0.05%-0.2% of metal chelating agent, 20%-25% of coating matrix, 4%-10% of coating solution, 0.5%-5% of enzyme for catalytic decomposition, 10%-15% of disintegrant, 40%-60% of forming agent, 0.5%-5% of sweetener, 0.3%-1% of flavoring agent and 0.1%-1% of lubricant.

2. The dental plaque indicator tablet according to claim 1, wherein the natural anthocyanin is selected from one or more of purple sweet potato anthocyanin, purple carrot anthocyanin, blueberry anthocyanin, elderberry anthocyanin, black wolfberry anthocyanin or purple cabbage extract.

3. The dental plaque indicator tablet according to claim 1, wherein the color auxiliary agent is selected from one or more of rosmarinic acid, chlorogenic acid, tannic acid, quercetin or catechin.

4. The dental plaque indicator tablet according to claim 1, wherein the antioxidant is selected from one or more of vitamin E, rosemary extract or ascorbyl palmitate; and the metal chelating agent is selected from one or more of ethylenediaminetetraacetic acid, ethylenediaminetetraacetic acid disodium salt or ethylenediaminetetraacetic acid tetrasodium salt.

5. The dental plaque indicator tablet according to claim 1, wherein the coating matrix is selected from one or more of cyclodextrin, pullulan, trehalose or polyvinyl alcohol; and the coating solution is selected from one or more of glycerol, polyethylene glycol, propylene glycol or sorbitol solution.

6. The dental plaque indicator tablet according to claim 1, wherein the enzyme for catalytic decomposition is selected from a composition of dextranase, allosteric enzyme and lysozyme, and a mass ratio of the dextranase to allosteric enzyme to lysozyme is 1.0-3.0:0.5-2.0:0.5-1.5.

7. The dental plaque indicator tablet according to claim 1, wherein the disintegrant is selected from one or more of crospovidone, microcrystalline cellulose or sodium carboxymethyl starch.

8. The dental plaque indicator tablet according to claim 1, wherein the forming agent is selected from one or more of glucose, polyethylene glycol, lactose, maltodextrin or sorbitol.

9. The dental plaque indicator tablet according to claim 1, wherein the sweetener is selected from one or more of sucralose, aspartame, ammonium glycyrrhizinate or xylitol; the flavoring agent is selected from peppermint flavor or fruit flavor; and the lubricant is magnesium stearate.

10. A method for preparing the dental plaque indicator tablet according to claim 1, comprising following steps:
step 1, mixing natural anthocyanin, color auxiliary agent, antioxidant, metal chelating agent, coating matrix and coating solution uniformly, performing vacuum drying until a water content is less than 5%, and a drying temperature is not more than 40 °C, crushing and sieving with an 80-mesh sieve to obtain a powder of natural anthocyanin stabilized color developing system;
step 2, uniformly mixing the powder obtained in the step 1 with the enzyme for catalytic decomposition, the disintegrant, the forming agent, the sweetener and the flavoring agent, and then adding the lubricant to continue mixing to obtain a total mixed powder; and
step 3, compressing the total mixed powder into tablets, and tableting parameters are as follows: tablet weight is 0.45 g-0.50 g, diameter is 10 mm-11 mm, thickness is 5.5 mm-6.0 mm, tablet shape is round, and hardness is 60 N-90 N; compressing environment temperature is 15 °C-25 °C, and relative humidity is below 45% RH.
